(19) United States
(12) Patent Application Publication
(10) Pub. No.: US 2007/0215466 A1
(43) Pub. Date: Sep. 20, 2007

(54) NUCLEIC ACID DETECTION DEVICE AND NUCLEIC ACID DETECTION APPARATUS

(76) Inventors: Jun Okada, Tokyo (JP); Sadato Hongo, Yokohama-shi (JP); Nobuhiro Gemma, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/555,936

(22) Filed: Nov. 2, 2006

(30) Foreign Application Priority Data
Mar. 17, 2006 (JP) 2006-075625

(51) Int. Cl. G01N 33/487

(52) U.S. Cl. 204/403.01

(57) ABSTRACT

A nucleic acid detection device includes a flow channel which allows a solution containing a nucleic acid recognition body to flow therethrough, at least two first electrode portions including a probe electrode to which at least one nucleic acid probe is immobilized, and at least two second electrode portions including a reference electrode to be used for measuring a reference value. The first and second electrode portions are arranged in the flow channel to form a row along the flow channel. One of the second electrode portions is adjacent to one of the first electrode portions.

### CROSS-REFERENCE TO RELATED APPLICATIONS

This application is based upon and claims the benefit of priority from prior Japanese Patent Application No. 2006-075625, filed Mar. 17, 2006, the entire contents of which are incorporated herein by reference.

### BACKGROUND OF THE INVENTION

1. Field of the Invention

The present invention relates to a nucleic acid detection device to detect a target nucleic acid by measurement through an electrochemical response which utilizes a nucleic acid recognition body, and a nucleic acid detection apparatus to measure the nucleic acid detection device.

2. Description of the Related Art

As genetic engineering develops in recent years, disease diagnosis or prevention using a gene has become possible in the medical field. This is called gene diagnosis. Detecting a human gene defect or change that causes a disease allows the disease to be diagnosed or predicted precritically or at its very early stage. Along with human genome decoding, researches on the relationship between the genotype and disease advance. Treatments that match the genotypes of the individual patients (tailor-made medical care) are being put into practice. Accordingly, it is very important to detect a gene and determine a genotype simply.

Examples of nucleic acid detection methods include a method using a radioisotope and a method using a fluorescent dye label. The former method can perform detection only at limited locations and requires cumbersome operation. The latter method requires an expensive apparatus to detect a fluorescent dye.

Besides these techniques, another technique has been established. According to this technique, a sample nucleic acid is hybridized with a nucleic acid probe immobilized to the surface of an electrode. Then, a nucleic acid recognition body is added and electrochemically detected. The technique of electrochemically detecting a nucleic acid is suitable to “Lab-on-a-chip” of causing reactions on a single chip. Hence, this technique has been under development in a variety of applications.

In a reaction on a chip with a comparatively small region, it is difficult to uniform the concentration distribution of nucleic acid recognition bodies within a nucleic acid probe immobilized region, and this degrades accuracy, unlike a reaction using a large-capacity cell. In particular, assume a device in which nucleic acid recognition bodies are supplied to a flow channel formed in a nucleic acid probe immobilized region. In this device, due to the concentration distribution of the nucleic acid recognition bodies from upstream to downstream in the flow channel, the accuracy decreases.

Techniques such as stirring of a solution by shaking or ultrasonic waves are also developed to uniform the concentration distribution. If such a technique is employed, however, the apparatus arrangement becomes complicated.

As described above, in the reaction on a chip, a decrease in accuracy due to the presence of the concentration distribution of the nucleic acid recognition bodies in the nucleic acid probe immobilized region poses a problem. Various types of nucleic acid recognition bodies are available. For example, a nucleic acid probe which has a sequence complimentary with a target nucleic acid hybridizes with the target nucleic acid to form a double stranded nucleic acid. A double stranded nucleic acid recognition body recognizes the double stranded nucleic acid and is strongly adsorbed by it. A nucleic acid probe which has a sequence uncomplimentary with the target nucleic acid does not hybridize with the target nucleic acid to stay in a single stranded state. The double stranded nucleic acid recognition body is weakly adsorbed by the single stranded nucleic acid probe. The double stranded nucleic acid recognition body is also adsorbed by an electrode surface where no nucleic acid exists. Assume that the electrochemical response of the nucleic acid recognition body is to be measured to detect the presence/absence of a target nucleic acid. In this case, whether the recognition body is adsorbed by a double stranded nucleic acid, a single stranded nucleic acid, or an electrode surface cannot be discriminated. Hence, in addition to the electrochemical response caused by hybridization of the target nucleic acid, a background electrochemical response (reference value, negative control) exists. This response is caused by adsorption to the single stranded nucleic acid or the electrode surface. This is regarded as the defect of the scheme that detects a nucleic acid by using the electrochemical response of the nucleic acid target body. This is based on the comparison with the scheme that detects a nucleic acid with a fluorescent dye. If the nucleic acid recognition body concentration is high, the background electrochemical response (reference value) increases. If the nucleic acid recognition body concentration is low, the electrochemical response caused by hybridization decreases. Hence, the nucleic acid recognition body concentration must be so set as to fall within an optimal concentration range. The nucleic acid recognition body, however, is strongly adsorbed by the flow channel wall surface or the support substrate as well. This decreases the nucleic acid recognition body concentration in the solution. Then, a concentration distribution is inevitably formed between the upstream and downstream of the flow channel. Furthermore, nucleic acid hybridization is easily influenced by the temperature, the salt concentration, the pH of the solution, the flow velocity of the solution, and the like. The nucleic acid recognition body should react in such a condition that a nucleic acid bond in which the target nucleic acid hybridizes with the nucleic acid probe will not be dissociated. These problems are specific to the technique of detecting a nucleic acid using the electrochemical response of nucleic acid recognition bodies which are supplied using a flow channel structure.

### BRIEF SUMMARY OF THE INVENTION

A nucleic acid detection device according to the present invention includes a flow channel which allows a solution containing a nucleic acid recognition body to flow therethrough, at least two first electrode portions including a probe electrode to which at least one nucleic acid probe is immobilized, and at least two second electrode portions including a reference electrode to be used for measuring a reference value. The first and second electrode portions are arranged in the flow channel to form a row along the flow channel, and one of the second electrode portions is adjacent to one of the first electrode portions.

Another nucleic acid detection device according to the present invention includes a flow channel which allows a solution containing a nucleic acid recognition body to flow therethrough, and electrode portions arranged in the flow channel to form a row along the flow channel. The electrode portions include a probe electrode to which at least one nucleic acid probe is immobilized and a reference electrode to be used for measuring a reference value.

Still another nucleic acid detection device according to the present invention includes a flow channel which allows a solution containing a nucleic acid recognition body to flow therethrough, at least two first electrode portions including a probe electrode to which at least one nucleic acid probe is immobilized, at least one second electrode portion including a reference electrode to be used for measuring a reference value, the first and second electrode portions being arranged in the flow channel to form a row along the flow channel, and at least two concentration measurement electrodes arranged in the flow channel to be used for measuring a relative value of a concentration distribution of the nucleic acid recognition body in the flow channel.

### BRIEF DESCRIPTION OF THE SEVERAL VIEWS OF THE DRAWING

FIG. 1 shows a nucleic acid detection device to be used for electrochemical nucleic acid detection;

FIG. 2 shows in enlargement the flow channel shown in FIG. 2;

FIG. 3 shows an electrode layout in a conventional nucleic acid detection device;

FIG. 4 shows the electrochemical response of a nucleic acid recognition body obtained for the nucleic acid detection device of FIG. 3;

FIG. 5 shows an electrode layout in a nucleic acid detection device according to the first embodiment;

FIG. 6 shows the electrochemical response of a nucleic acid recognition body obtained for the nucleic acid detection device of FIG. 5;

FIG. 7 shows an electrode layout in a nucleic acid detection device according to the second embodiment;

FIG. 8 shows the electrochemical response of a nucleic acid recognition body obtained for the nucleic acid detection device of FIG. 7;

FIG. 9 shows an electrode layout in a nucleic acid detection device according to the third embodiment;

FIG. 10 shows the electrochemical response of a nucleic acid recognition body obtained for the nucleic acid detection device of FIG. 9;

FIG. 11 shows an electrode layout in a nucleic acid detection device according to the fourth embodiment;

FIG. 12 shows the electrochemical response of a nucleic acid recognition body obtained for the nucleic acid detection device of FIG. 11;

FIG. 13 shows an electrode layout in a nucleic acid detection device according to the fifth embodiment;

FIG. 14 shows the electrochemical response of a nucleic acid recognition body obtained for the nucleic acid detection device of FIG. 13;

FIG. 15 shows an electrode layout in a nucleic acid detection device according to the sixth embodiment;

FIG. 16 shows the electrochemical response of a nucleic acid recognition body obtained for the nucleic acid detection device of FIG. 15;

FIG. 17 shows an electrode layout in a nucleic acid detection device according to the seventh embodiment;

FIG. 18 shows the electrochemical response of a nucleic acid recognition body obtained for the nucleic acid detection device of FIG. 17;

FIG. 19 shows an electrode layout in a nucleic acid detection device according to the eighth embodiment; and

FIG. 20 shows the electrochemical response of a nucleic acid recognition body obtained for the nucleic acid detection device of FIG. 19.

### DETAILED DESCRIPTION OF THE INVENTION

The embodiments of the present invention will be described with reference to the accompanying drawing.

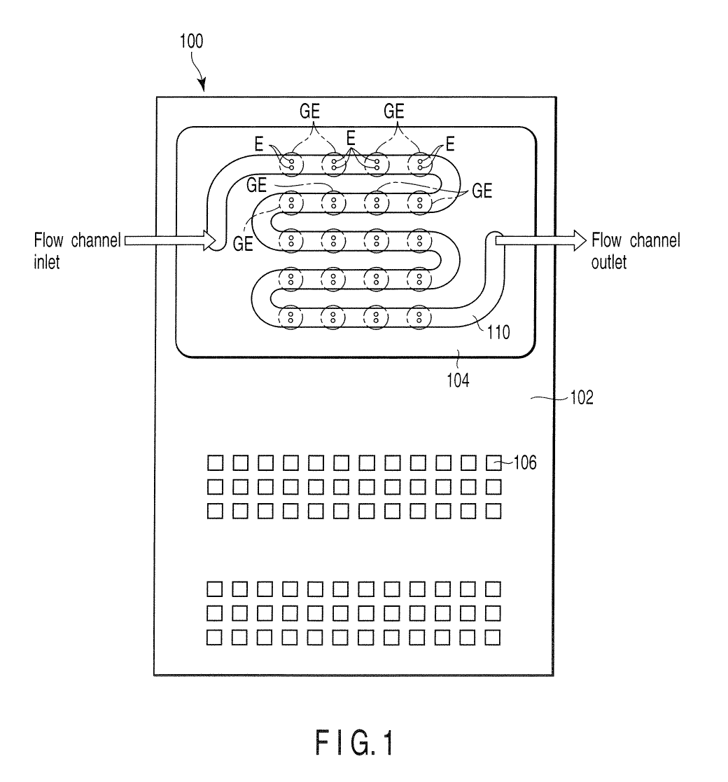

As shown in FIG. 1, a nucleic acid detection device 100 has a support substrate 102 and a flow channel regulation member 104. The support substrate 102 has signal input/output pads 106. The flow channel regulation member 104 has a flow channel 110 and 20 electrode portions GE. The flow channel 110 allows a solution containing nucleic acid recognition bodies to flow through it. The 20 electrode portions GE are arranged in the flow channel 110 and apart from each other so as to form a row along the flow channel 110.

Although each electrode portion GE comprises two electrodes E as shown in, e.g., FIG. 1, it is not limited to this. Each electrode portion GE may comprise three or more electrodes, or one electrode. The upper limit of the number of electrodes is, e.g., 10 or less. Preferably, the number of electrodes falls within a range of 1 to 4 (both inclusive).

The shape and the size of the electrode portion GE are not particularly limited. For example, if the region of the electrode portion GE forms a circle, it can fall within a range of 0.002 mm to 1.6 mm (both inclusive) and preferably 0.01 mm to 0.8 mm (both inclusive). If the region of the electrode portion GE forms a rectangle, the length of its one side can fall within a range of 0.002 mm to 1.6 mm (both inclusive) and preferably 0.01 mm to 0.8 mm (both inclusive).

The shape and the size of each electrode exposed to the flow channel in the electrode portion GE are not particularly specified. For example, if the electrode forms a circle, its diameter can fall within a range of 0.001 mm to 0.8 mm (both inclusive) and preferably 0.001 mm to 0.4 mm (both inclusive). If the electrode forms a rectangle, the length of its one side can fall within a range of 0.001 mm to 0.8 mm (both inclusive) and preferably 0.001 mm to 0.4 mm (both inclusive).

The signal input/output pads 106 are electrically connected to the electrodes E in the flow channel 110, respectively. The number of electrode portions GE is not limited to the number of electrodes E but can be arbitrary. The interval among the respective electrode portions GE is not particularly specified, and can fall within a range of, e.g., 0.1 mm to 3.0 mm (both inclusive) and preferably 0.5 mm to 2.5 mm (both inclusive).

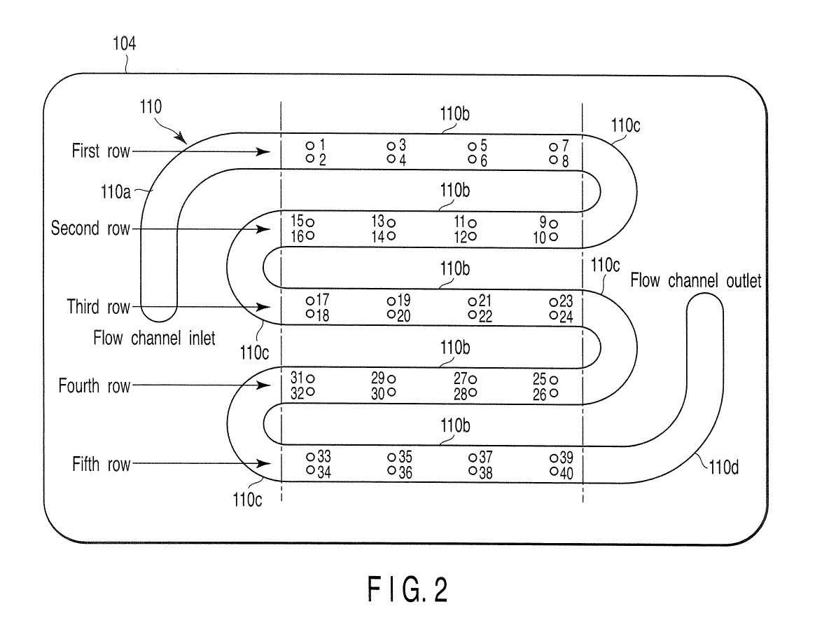

As shown in FIG. 2, the flow channel 110 comprises a flow channel inlet 110a, a flow channel outlet 110d, straight flow channel regions 110b, and semicircular connection flow channel regions 110c. The flow channel inlet 110a is located at the upstream end of the flow channel 110. The flow channel outlet 110d is located at the downstream end of the flow channel 110. The straight flow channel regions 110b are located between the flow channel inlet 110a and the flow channel outlet 110d. Each connection flow channel region 110c connects two adjacent straight flow channel regions 110b. The straight flow channel regions 110b are arrayed like rows. The connection flow channel regions 110c which connect the straight flow channel regions 110b need not be semicircular but can form, e.g., curves, or shapes of bent straight lines.

The electrode portions GE are arranged in the straight flow channel regions 110b. Although the electrode portions GE are arranged equidistantly in each straight flow channel region 110b, they need not always be arranged in this manner but can be arranged at irregular intervals.

The nucleic acid detection device 100 is used as it is mounted in a known nucleic acid detection apparatus. The nucleic acid detection apparatus supplies a solution containing nucleic acid recognition bodies to the flow channel 110 and measures the current flowing through the electrodes E of the electrode portions GE through the signal input/output pads 106.

The nucleic acid detection device 100 is not limited to the form shown in FIG. 1.

The flow channel 110 may be formed by forming a groove in the flow channel regulation member 104 and stacking a flat member on the flow channel regulation member 104. Alternatively, the flow channel 110 may be formed by forming a groove in that portion of a flat member which corresponds to the flow channel 110 and stacking the flat member on the flow channel regulation member 104. The flow channel 110 may also be formed by forming grooves in both a flat member and the flow channel regulation member 104 and stacking them. The flow channel regulation member 104 may not be employed, and the flow channel 110 may be directly formed in the support substrate 102. The sectional structure of the flow channel 110 may have an arbitrary shape, e.g., a polygon such as a square or a triangle, a semicircle, an ellipse, or the like.

For example, according to a practical example, the width of the flow channel of the straight flow channel region 110b and that of the connection flow channel region 110c can fall within a range of 0.05 mm to 3.0 mm (both inclusive) and preferably 0.2 mm to 1.5 mm (both inclusive). The height of the flow channel of the straight flow channel region 110b and that of the connection flow channel region 110c can fall within a range of 0.02 mm to 2.0 mm (both inclusive) and desirably 0.1 mm to 1.2 mm (both inclusive). If the flow channel size falls within these ranges, variations in concentration of nucleic acid recognition bodies in the flow channel can be suppressed.

The width of the flow channel 110 is always constant, but may vary to change from wide to narrow, narrow to wide, and wide to narrow repeatedly.

The signal input/output pads 106 on the support substrate 102 may be omitted when necessary. The nucleic acid detection device 100 may comprise a region to perform various types of reaction steps and other detections such as a nucleic acid extraction reaction, a nucleic acid amplification reaction, a filtration step, and a stirring step.

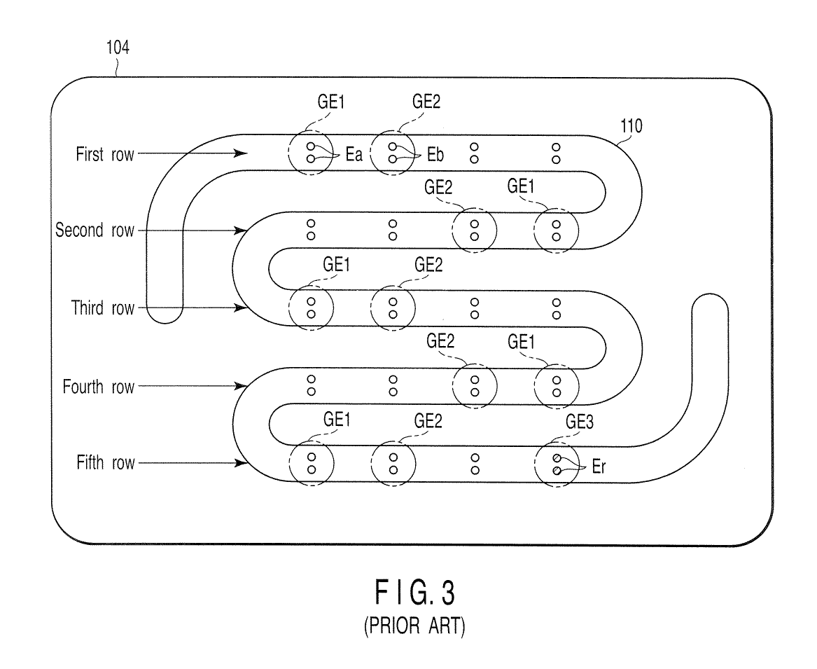

FIGS. 3, 5, 7, 9, 11, 13, 15, 17, and 19, which are referred to when describing the following conventional example and the embodiments, show the electrode layout. For the sake of descriptive convenience, electrode numbers shown in FIG. 2 are assigned to the electrodes of the electrode portions on the flow channel of each view of the drawing. Counting from the flow channel inlet side, electrodes in the first-row straight flow channel region will be defined as the 1st to 8th electrodes, electrodes in the second-row straight flow channel region will be defined as the 9th to 16th electrodes, and the like.

### CONVENTIONAL EXAMPLE

In a conventional nucleic acid detection device, as shown in FIG. 3, in the first-row straight flow channel region, the 1st and 2nd electrodes are probe electrodes to each of which at least one nucleic acid probe having a sequence complimentary to the target nucleic acid is immobilized. The 3rd and 4th electrodes are probe electrodes to each of which at least one nucleic acid probe having a sequence uncomplimentary to the target nucleic acid is immobilized. This applies to the straight flow channel regions from the second row. The 39th and 40th electrodes located at the terminal end of a flow channel 110 are reference electrodes to be used for reference value measurement.

In other words, in each straight flow channel region, an electrode portion GE1 which is located on the most upstream side has two probe electrodes Ea to each of which nucleic acid probes (complementary sequence) are immobilized. An electrode portion GE2 which is located next has two probe electrodes Eb to each of which nucleic acid probes (uncomplimentary sequence) are immobilized. An electrode portion GE3 which is located on the most downstream side in the entire flow channel 110 has two reference electrodes Er.

In the nucleic acid detection device of FIG. 3, a value measured through the reference electrodes of the 39th and 40th electrodes is employed as a reference value for the nucleic acid probes arranged at the 1st to 4th electrodes, 9th to 12th electrodes, . . . . The reference value is compared with a current value obtained through the probe electrodes, so that the presence/absence of the target nucleic acid is detected, and its quantitation is analyzed.

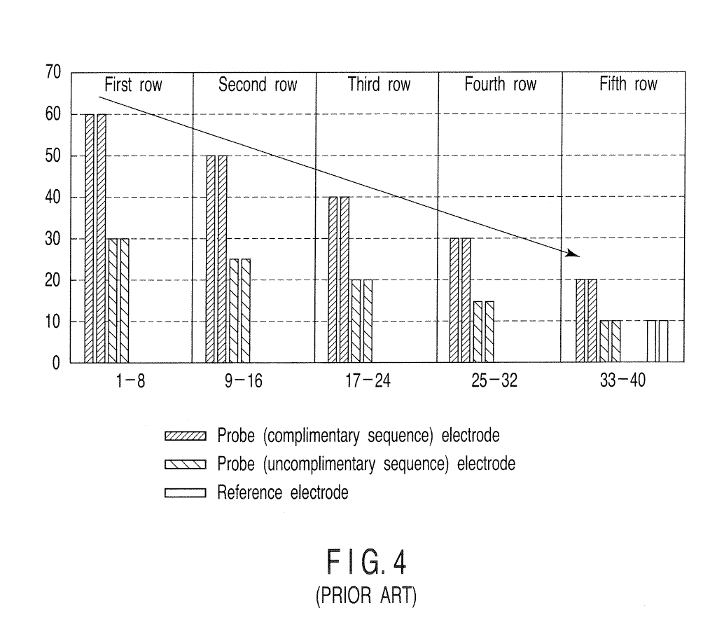

As is apparent from FIG. 4, the electrochemical response gradually decreases as the flow advances from the first row to the fifth row. Normally, the reference electrodes Er and the probe electrodes Eb should show almost the same values, as in the result of the fifth row. In the other rows, however, the electrochemical response at the probe electrodes Eb is high, and highly accurate nucleic acid detection cannot be achieved.

### First Embodiment

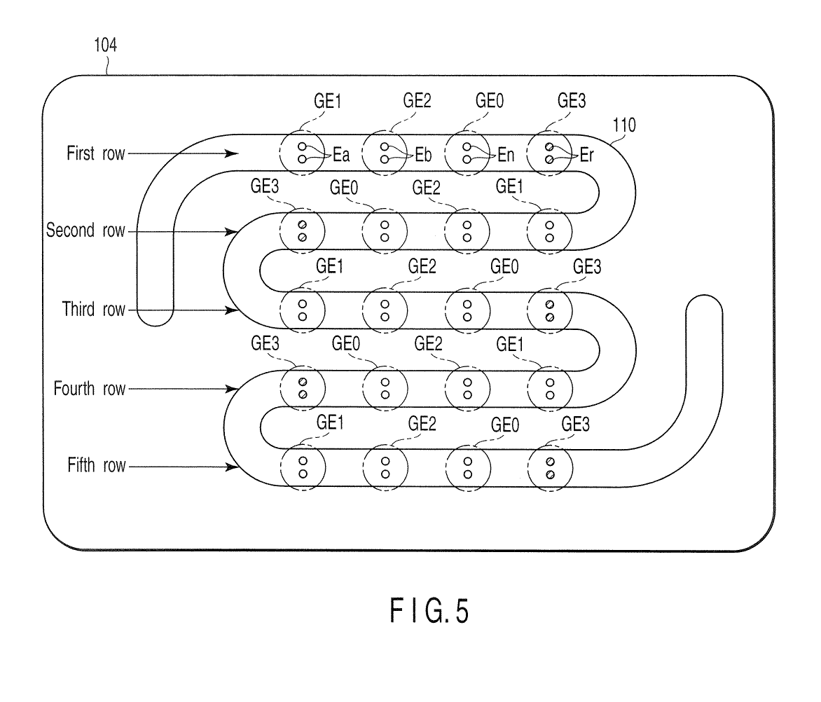

In a nucleic acid detection device according to the first embodiment, as shown in FIG. 5, in the first-row straight flow channel region, the first and second electrodes are probe electrodes to each of which at least one nucleic acid probe having a sequence complimentary to the target nucleic acid is immobilized. The third and fourth electrodes are probe electrodes to each of which at least one nucleic acid probe having a sequence uncomplimentary to the target nucleic acid is immobilized. The seventh and eighth electrodes are reference electrodes Er. This applies to the straight flow channel regions from the second row.

The reference electrode Er is formed by immobilizing, e.g., a reference value measurement nucleic acid probe. The reference value measurement nucleic acid probe has a sequence that is not included in the sample nucleic acid, or a sequence complimentary to this. The reference electrode Er is not limited to one obtained by immobilizing a reference value measurement nucleic acid probe to an electrode, but may comprise an electrode to which no nucleic acid probe is immobilized. For example, a normal chain organic molecule, or an aromatic molecule, a synthesized nucleic acid, or an enzyme may be immobilized. As the step of immobilizing the reference value measurement nucleic acid probe, sometimes a step of dropping a trace of solution containing a reference value measurement nucleic acid probe onto the electrode is necessary. Hence, the electrode-to-electrode distance cannot be decreased to be smaller than the diameter of the drop to be dropped, so that the device area cannot be decreased and the necessary sample amount cannot be decreased. However, if the nucleic acid probe is not immobilized unlike in the above case, a reference electrode can be formed by the step of dipping the entire device in a solution containing various types of molecules. Therefore, the electrode-to-electrode distance can be decreased, leading to a low cost. The value of the current which is indicated when the target nucleic acid does not hybridize with the probe electrode and the current value indicated by the reference electrode value should be equal, or should be easily comparable.

Each straight flow channel region includes four electrode portions arranged with the same sequence pattern. In each straight flow channel region, an electrode portion GE1 which is located on the most upstream side has two probe electrodes Ea to each of which nucleic acid probes (complementary sequence) are immobilized. An electrode portion GE2 which is located on the second upstream side has two probe electrodes Eb to each of which nucleic acid probes (uncomplimentary sequence) are immobilized. An electrode portion GE0 which is located on the third upstream side has two electrodes En which have no special function. An electrode portion GE3 which is located on the most downstream side has the two reference electrodes Er. Therefore, in an entire flow channel 110, the electrode portions are repeatedly arranged with a sequence pattern of GE1, GE2, GE0, and GE3 along the flow channel 110. Regarding the electrode portions GE1 and GE3, they are adjacent and arrayed alternately. The distinction among the electrode portions GE1, GE2, GE0, and GE3 is based on the difference in function among the probe electrodes Ea, Eb, En, and Er included in them. The probe electrodes Ea and Eb have the same function in the respect that nucleic acid probes are immobilized to them. However, the nucleic acid probes of the probe electrodes Ea and Eb have different nucleic acid sequences. Thus, the probe electrodes Ea and Eb detect nucleic acids having difference sequences and accordingly have different functions.

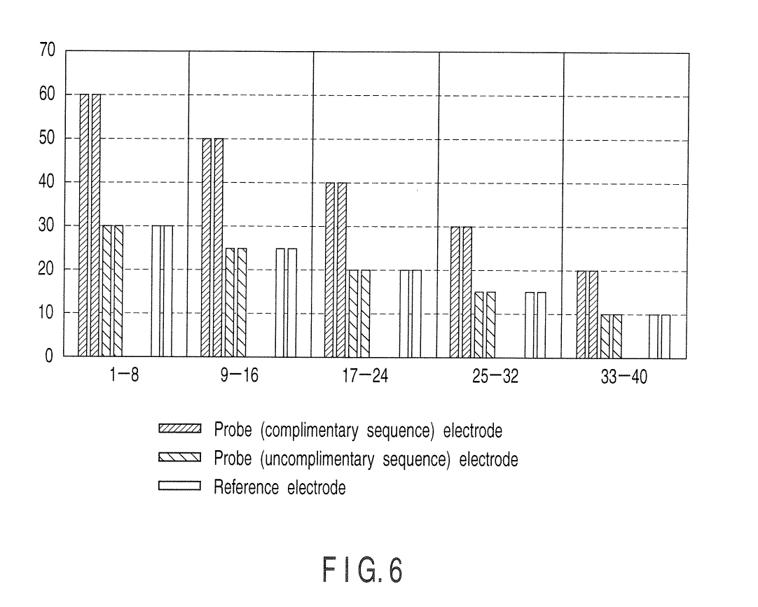

In the electrochemical response shown in FIG. 6, regarding the first-row straight flow channel region, a value measured through the 7th and 8th reference electrodes is employed as a reference value for the 1st to 4th probe electrodes. Regarding the second-row straight flow channel region, a value measured through the 15th and 16th reference electrodes is employed as a reference value for the 9th to 12th probe electrodes. This applies to the straight flow channel regions from the third row. In the straight flow channel region of each row, a current value obtained through the probe electrode is compared with a reference value based on the reference electrode in the straight flow channel region of the same row. Thus, the presence/absence of the target nucleic acid is detected, and its quantitation is analyzed.

With this arrangement, a highly accurate reference value that reflects the concentration distribution of the nucleic acid recognition bodies in each row can be measured. When the value of the nucleic acid probe is compared with the reference value, highly accurate detection can be performed.

### Second Embodiment

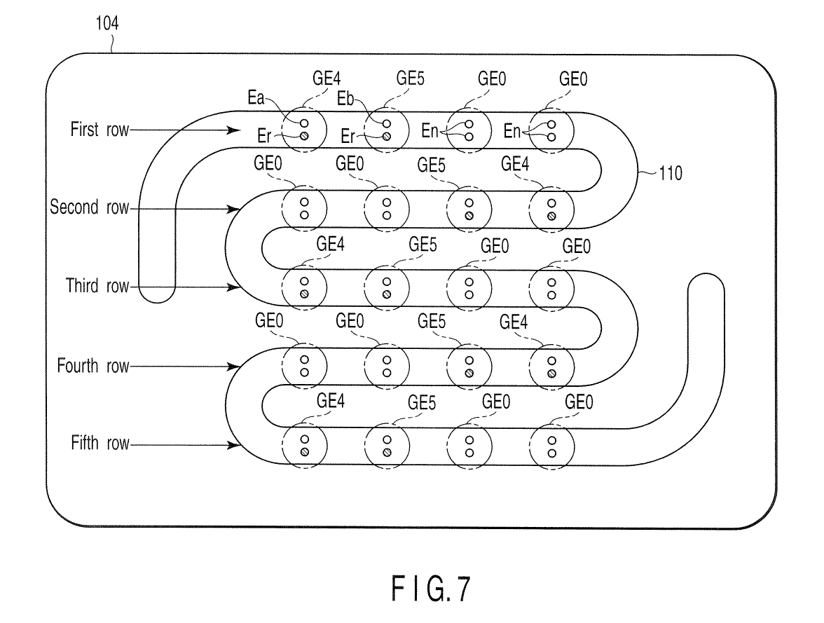

In a nucleic acid detection device according to the second embodiment, as shown in FIG. 7, in the first-row straight flow channel region, the first electrode is a probe electrode to which a nucleic acid probe having a sequence complimentary to a target nucleic acid is immobilized. The third electrode is a probe to which a nucleic acid probe having a sequence uncomplimentary to the target nucleic acid is immobilized. The second and fourth electrodes are reference electrodes which are used for measuring a reference value. This applies to the straight flow channel regions from the second row.

In other words, each straight flow channel region includes four electrode portions arranged with the same sequence pattern. In each straight flow channel region, an electrode portion GE4 which is located on the most upstream side has one probe electrode Ea to which nucleic acid probes (complementary sequence) are immobilized and one reference electrode Er. An electrode portion GE5 which is located next has one probe electrode Eb to which nucleic acid probes (uncomplimentary sequence) are immobilized and one reference electrode Er. Each remaining electrode portion GE0 has two electrodes En which have no special function. Therefore, in an entire flow channel 110, the electrode portions are repeatedly arranged with a sequence pattern of GE4, GE5, GE0, and GE0 along the flow channel 110.

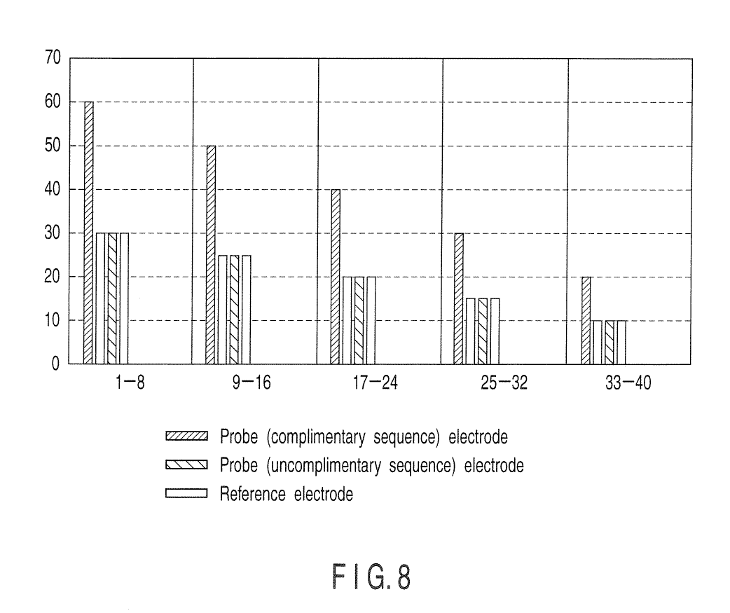

In the electrochemical response shown in FIG. 8, regarding the first-row straight flow channel region, a value measured through the second reference electrode is employed as a reference value for the first probe electrode. A value measured through the third reference electrode is employed as a reference value for the fourth probe electrode. The reference value is compared with a current value obtained through the probe electrode. Thus, the presence/absence of the target nucleic acid is detected, and its quantitation is analyzed. This applies to the straight flow channel regions from the second row.

Namely, according to this embodiment, a current value obtained through a probe electrode is compared with the reference value based on a value measured through a reference electrode which is included in an electrode portion that includes this probe electrode.

With this arrangement, a highly accurate reference value that reflects the concentration distribution of the nucleic acid recognition bodies at the position of each nucleic acid detection electrode can be measured. When the value of the nucleic acid probe is compared with the reference value, highly accurate detection can be performed.

### Third Embodiment

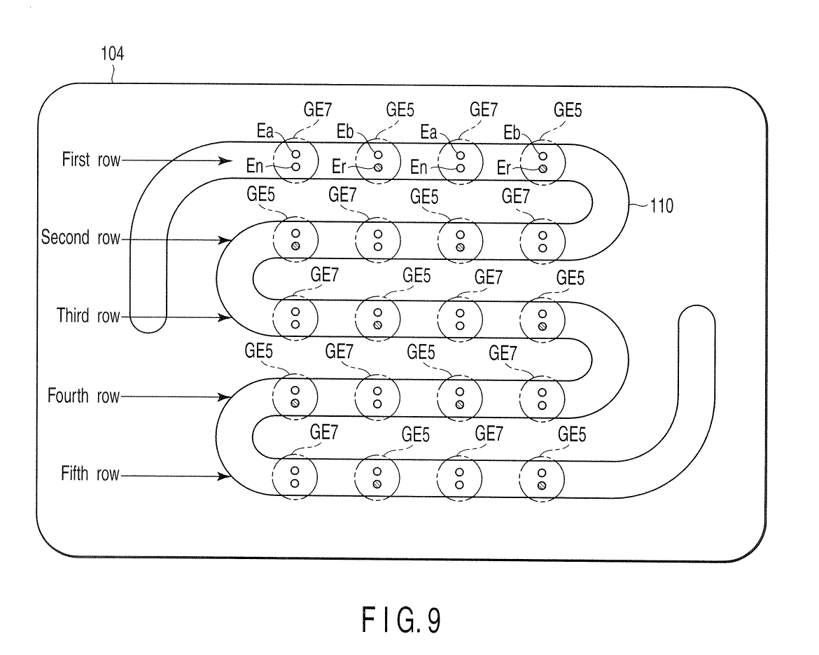

In a nucleic acid detection device according to the third embodiment, as shown in FIG. 9, in the first-row straight flow channel region, the first and fifth electrodes are probe electrodes to each of which at least one nucleic acid probe having a sequence complimentary to a target nucleic acid is immobilized. The third and seventh electrodes are probe electrodes to each of which at least one nucleic acid probe having a sequence uncomplimentary to the target nucleic acid is immobilized. The fourth and eighth electrodes are reference electrodes which are used for measuring a reference value. This applies to the straight flow channel regions from the second row.

In other words, each straight flow channel region includes four electrode portions arranged with the same sequence pattern. In each straight flow channel region, each of electrode portions GE7 which are located the first and third from the upstream side has one probe electrode Ea to which nucleic acid probes (complementary sequence) are immobilized and a reference electrode Er. Each of electrode portions GE5 which are located the second and fourth from the upstream side has one probe electrode Eb to which nucleic acid probes (uncomplimentary sequence) are immobilized and one reference electrode Er. Therefore, in an entire flow channel 110, the electrode portions are repeatedly arranged with a sequence pattern of GE7, GE5, GE7, and GE5 along the flow channel 110.

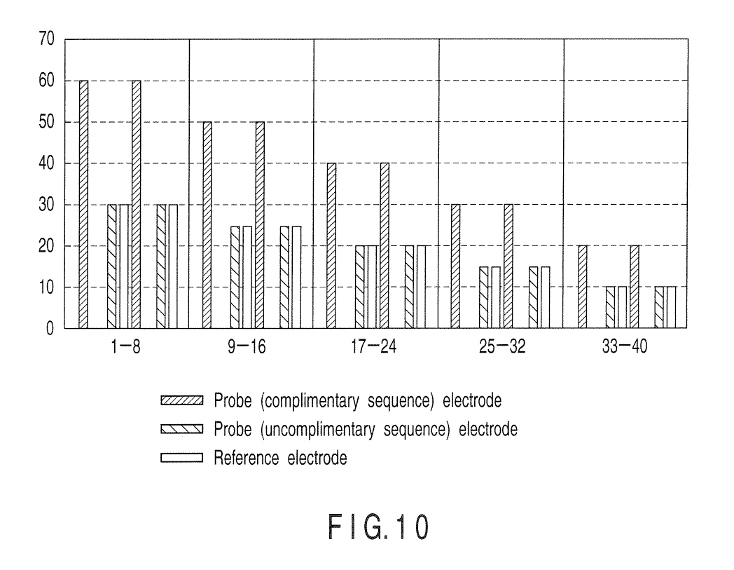

In the electrochemical response shown in FIG. 10, regarding the first-row straight flow channel region, a value measured through the fourth reference electrode is employed as a reference value for the first and third probe electrodes. A value measured through the eighth reference electrode is employed as a reference value for the fifth and seventh probe electrodes. The reference value is compared with a current value obtained through the probe electrode. Thus, the presence/absence of the target nucleic acid is detected, and its quantitation is analyzed. This applies to the straight flow channel regions from the second row.

Namely, according to this embodiment, a current value obtained through a probe electrode is compared with the reference value based on a value measured through a reference electrode which is included in an electrode portion that includes this probe electrode, or an adjacent electrode portion.

With this arrangement, in each row, a highly accurate reference value that reflects the concentration distribution of the nucleic acid recognition bodies at the two, intermediate and terminal end points can be measured. When the value of the nucleic acid probe is compared with the reference value, highly accurate detection can be performed.

### Fourth Embodiment

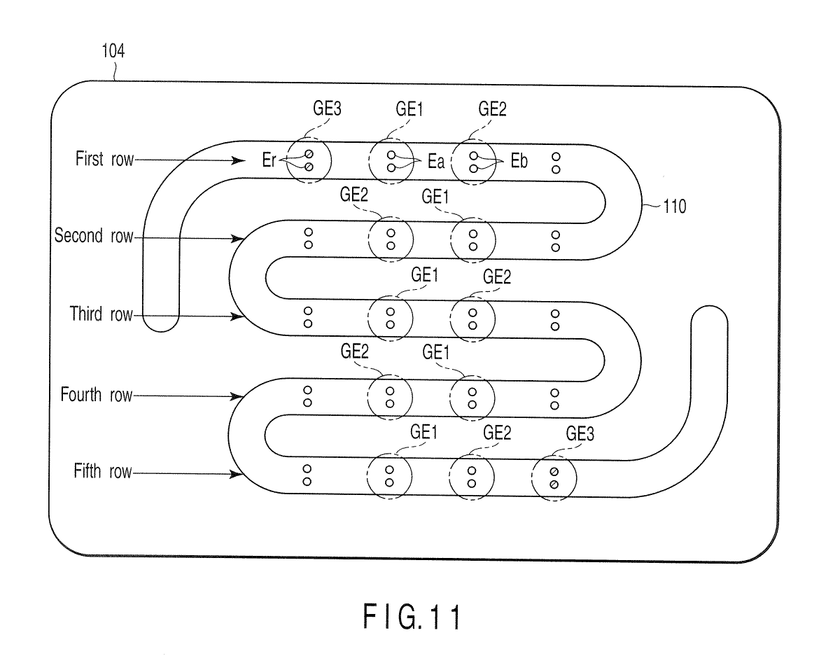

In a nucleic acid detection device according to the fourth embodiment, as shown in FIG. 11, in the first-row straight flow channel region, the 3rd and 4th electrodes are probe electrodes to each of which at least one nucleic acid probe having a sequence complimentary to a target nucleic acid is immobilized. The 5th and 6th electrodes are probe electrodes to each of which at least one nucleic acid probe having a sequence uncomplimentary to the target nucleic acid is immobilized. This applies to the straight flow channel regions from the second row. The 1st, 2nd, 39th, and 40th electrodes are reference electrodes which are used for measuring a reference value.

In other words, in each straight flow channel region, an electrode portion GE1 which is located the second from the upstream side has two probe electrodes Ea to each of which nucleic acid probes (complementary sequence) are immobilized. An electrode portion GE2 which is located the third from the upstream side has two probe electrodes Eb to each of which nucleic acid probes (uncomplimentary sequence) are immobilized. In an entire flow channel 110, each of an electrode portion GE3 which is located on the most upstream side and an electrode portion GE3 which is located on the most downward side has two reference electrodes Er. In other words, the electrode portions GE1 and GE2 including the probe electrodes Ea and Eb, respectively, are located between the electrode portions GE3 each including the reference electrodes Er.

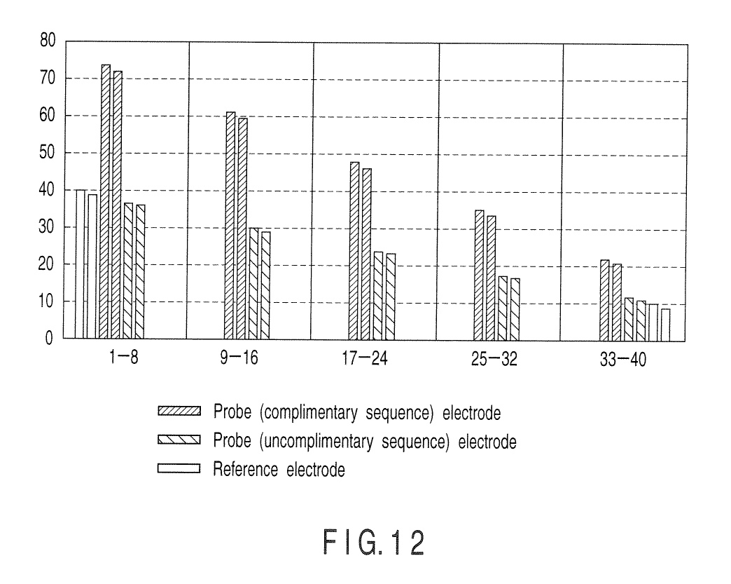

In the electrochemical response shown in FIG. 12, a value calculated on the basis of a value measured through the 1st, 2nd, 39th, and 40th reference electrodes is employed as a reference value for all the probe electrodes. The reference value is compared with a current value obtained through the probe electrode. Thus, the presence/absence of the target nucleic acid is detected, and its quantitation is analyzed. The reference value can be calculated by various equations depending on the arrangement of the device. For example, the reference value can be calculated on the basis of the distance between the reference electrode and a nucleic acid probe, or along a calibration curve prepared in advance. In addition, a known appropriate arbitrary calculation method can be employed.

With this arrangement, in each row, a highly accurate reference value that reflects the concentration distribution of the nucleic acid recognition bodies at each electrode position can be measured. When the value of the nucleic acid probe is compared with the reference value, highly accurate detection can be performed.

### Fifth Embodiment

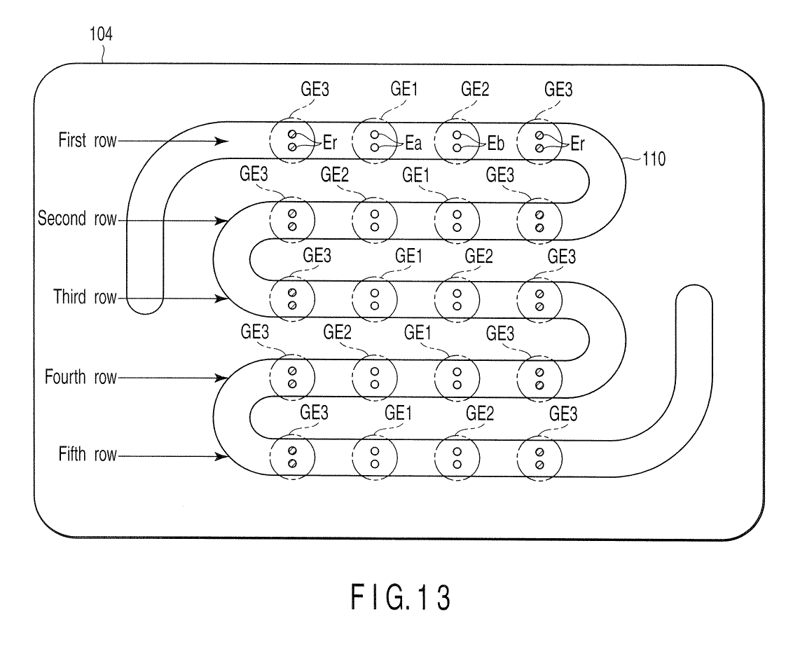

In a nucleic acid detection device according to the fifth embodiment, as shown in FIG. 13, in the first-row straight flow channel region, the third and fourth electrodes are probe electrodes to each of which at least one nucleic acid probe having a sequence complimentary to a target nucleic acid is immobilized. The fifth and sixth electrodes are probe electrodes to each of which at least one nucleic acid probe having a sequence uncomplimentary to the target nucleic acid is immobilized. First, second, seventh, and eighth electrodes are reference electrodes which are used for measuring a reference value. This applies to the straight flow channel regions from the second row.

In other words, each straight flow channel region includes four electrode portions arranged with the same sequence pattern. In each straight flow channel region, each of electrode portions GE3 which are located the first and fourth from the upstream side has two reference electrodes Er. An electrode portion GE1 which is located the second from the upstream side has two probe electrodes Ea to each of which nucleic acid probes (complimentary sequence) are immobilized. An electrode portion GE2 which is located the third from the upstream side has two probe electrodes Eb to each of which nucleic acid probes (uncomplimentary sequence) are immobilized. Therefore, in an entire flow channel 110, the electrode portions are repeatedly arranged with the sequence pattern of GE3, GE1, GE2, and GE3 along the flow channel 110.

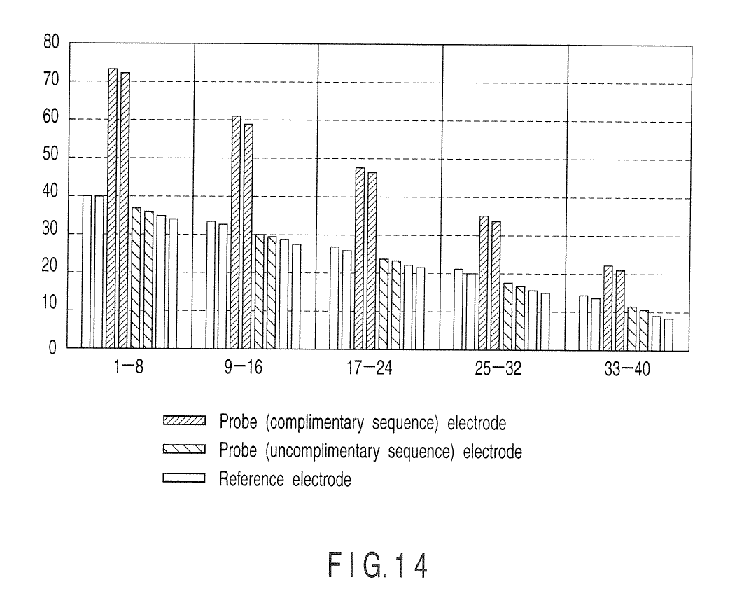

In the electrochemical response shown in FIG. 14, regarding the first-row straight flow channel region, a value calculated on the basis of a value measured through the 1st, 2nd, 7th, and 8th reference electrodes is employed as a reference value for the 3rd to 6th probe electrodes. Regarding the second-row straight flow channel region, a value measured through the 9th, 10th, 15th, and 16th reference electrodes is employed as a reference value for the 11th to 14th probe electrodes. This applies to the straight flow channel regions from the third row. In this manner, in the straight flow channel region of each row, the reference value is based on the reference electrode in the straight flow channel region of this row. A current value obtained through the probe electrode is compared with the reference value. Thus, the presence/absence of the target nucleic acid is detected, and its quantitation is analyzed. The reference value can be calculated by a known appropriate arbitrary calculation method, as described in the fourth embodiment.

With this arrangement, in each row, a highly accurate reference value that reflects the concentration distribution of the nucleic acid recognition bodies at each electrode position can be measured. When the value of the nucleic acid probe is compared with the reference value, highly accurate detection can be performed.

### Sixth Embodiment

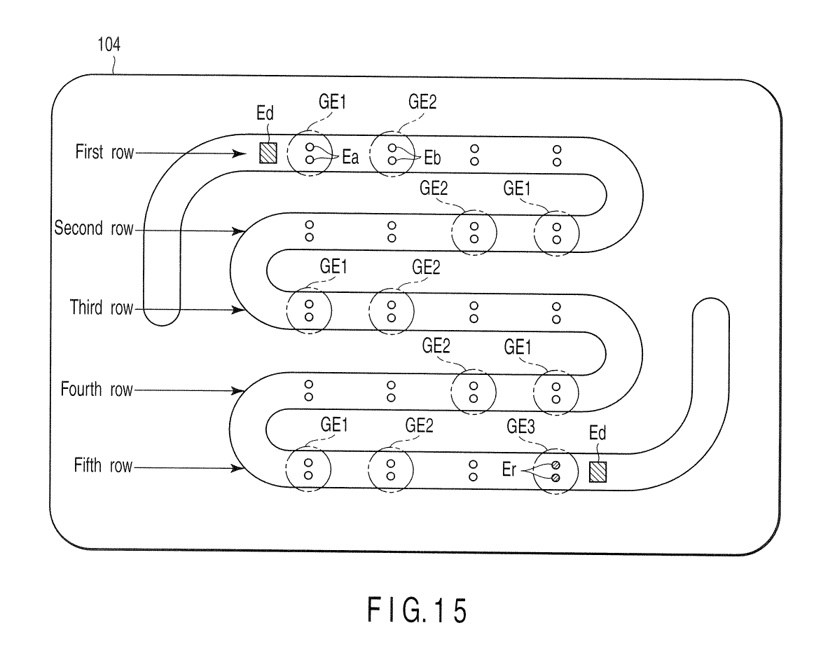

In a nucleic acid detection device according to the sixth embodiment, as shown in FIG. 15, in the first-row straight flow channel region, the 1st and 2nd electrodes are probe electrodes to each of which at least one nucleic acid probe having a sequence complimentary to a target nucleic acid is immobilized. The 3rd and 4th electrodes are probe electrodes to each of which at least one nucleic acid having a sequence uncomplimentary to the target nucleic acid is immobilized. This applies to the straight flow channel regions from the second row. The 39th and 40th electrodes at the terminal end of a flow channel 110 are reference electrodes which are used for measuring a reference value. Furthermore, concentration measurement electrodes Ed used for measuring the concentration of the nucleic acid recognition bodies are arranged respectively on the flow channel upstream side of the 1st and 2nd electrodes and on the flow channel downstream side of the 39th and 40th electrodes. Each concentration measurement electrode Ed may have a clean surface to which nothing is adsorbed, or a surface to which halogen atoms such as Cl, Br, or I atoms are adsorbed. Alternatively, the surface of each concentration measurement electrode Ed may be formed of normal chain organic molecules with the terminal ends modified by thiol groups, or molecules with terminal ends modified by amino groups. The normal chain organic molecules can be molecules of mercaptoethanol, mercaptohexanol, or mercaptooctanol. The concentration measurement electrodes are not particularly limited as far as they indicate signals that depend on the concentration of the nucleic acid recognition bodies. For example, a normal chain organic molecule, an aromatic molecule, a synthesized nucleic acid, or an enzyme may be immobilized to each concentration measurement electrode. It is preferable if a molecule that interacts with the nucleic acid recognition body, or a molecule modified by a functional group that interacts with the nucleic acid recognition body is immobilized. Regarding the step of forming the concentration measurement electrode, a step of dipping the entire device in a solution containing various types of molecules can form the concentration measurement electrode. Thus, the electrode-to-electrode distance can be shortened to lead to a low cost. Unlike the reference electrode, the current value indicated when the target nucleic acid does not hybridize with the probe electrode and the current value indicated by the concentration measurement electrode need not be equal, or need not easily comparable. Thus, limitations on the electrode area and position are reduced. For example, assume that the diameters of the probe electrodes are 200 μm and the electrode interval is 2 mm. The concentration measurement electrodes have diameters of 50 μm and can be arranged in the 2-mm gap between the probe electrodes. Thus, the device can be formed at low cost without occupying an extra region.

In other words, in each straight flow channel region, an electrode portion GE1 which is located on the most upstream side has two probe electrodes Ea to each of which nucleic acid probes (complementary sequence) are immobilized. An electrode portion GE2 which is located the second upstream side has two probe electrodes Eb to each of which nucleic acid probes (uncomplimentary sequence) are immobilized. In the entire flow channel 110, an electrode portion GE3 which is located on the most downstream side has two reference electrodes Er. The electrode portions GE1, GE2, and GE3 are located between concentration measurement electrodes Ed.

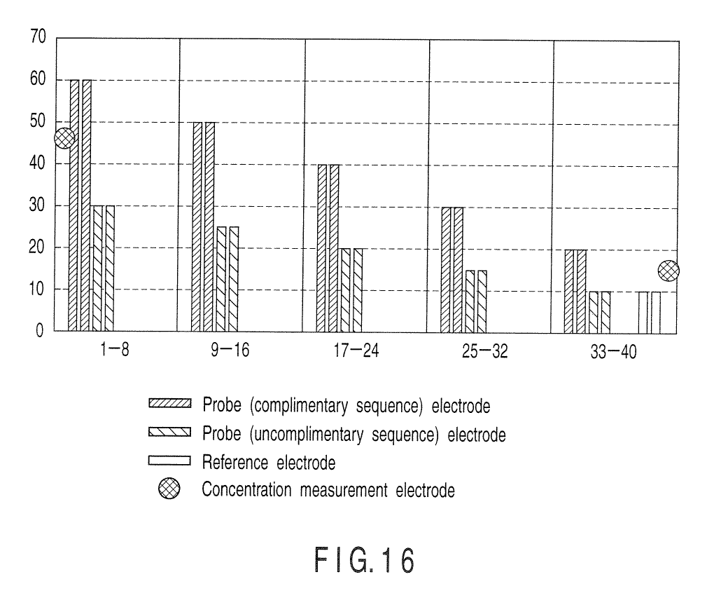

In the electrochemical response shown in FIG. 16, a current value obtained through all or some probe electrodes and all or some reference electrodes is corrected using a value which is calculated on the basis of a value measured through the concentration measurement electrodes Ed. The corrected current value is used to check the presence/absence of the target nucleic acid and to determine its amount. The current value can be corrected by various equations depending on the arrangement of the device. For example, the current value can be corrected on the basis of the distance between the concentration measurement electrode and the nucleic acid probe, or the distance between the reference electrodes. Alternatively, the current value can be corrected along a calibration curve prepared in advance. In addition, a known appropriate arbitrary calculation method can be employed.

With this arrangement, in the flow channel, the current value can be corrected to reflect the concentration distribution of the nucleic acid recognition bodies at each electrode position. When the value of the nucleic acid probe is compared with the corrected current value, highly accurate detection can be performed.

### Seventh Embodiment

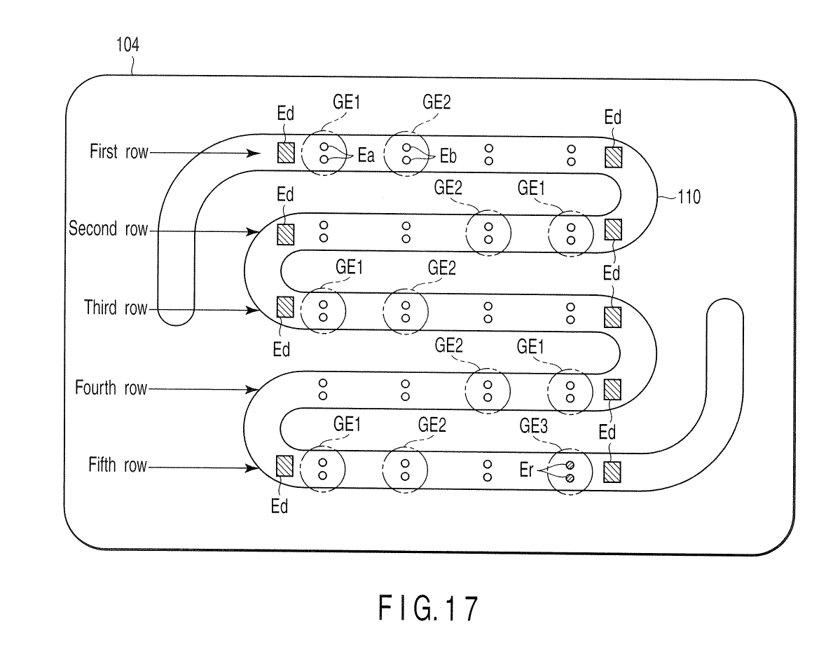

In a nucleic acid detection device according to the seventh embodiment, as shown in FIG. 17, in the first-row straight flow channel region, the 1st and 2nd electrodes are probe electrodes to each of which at least one nucleic acid probe having a sequence complimentary to a target nucleic acid is immobilized. The 3rd and 4th electrodes are probe electrodes to each of which at least one nucleic acid probe having a sequence uncomplimentary to the target nucleic acid is immobilized. Concentration measurement electrodes are arranged on the flow channel upstream side of the 1st and 2nd electrodes and on the flow channel downstream side of the 7th and 8th electrodes. This applies to the straight flow channel regions from the second row. The 39th and 40th electrodes at the terminal end of a flow channel 110 are reference electrodes which are used for measuring a reference value.

In other words, in each straight flow channel region, an electrode portion GE1 which is located on the most-upstream side has two probe electrodes Ea to each of which nucleic acid probes (complimentary sequence) are immobilized. An electrode portion GE2 which is located the second upstream side has two probe electrodes Eb to each of which nucleic acid probes (uncomplimentary sequence) are immobilized. The electrode portions GE1 and GE2 are located between concentration measurement electrodes Ed. An electrode portion GE3 which is located on the most downstream side in the entire flow channel 110 has two reference electrodes Er.

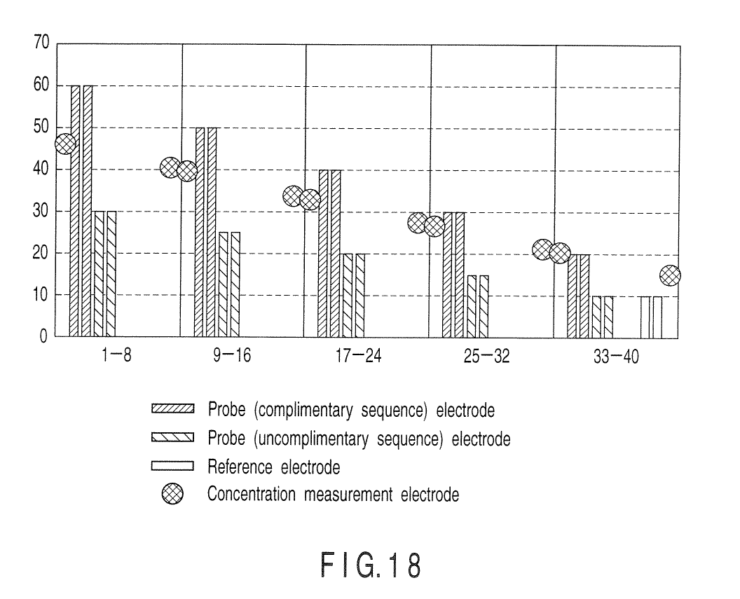

In the electrochemical response shown in FIG. 18, regarding the first-row straight flow channel region, a value calculated on the basis of values measured through the concentration measurement electrode arranged on the flow channel upstream side of the first and second electrodes and through the concentration measurement electrode arranged on the flow channel downstream side of the seventh and eighth electrodes is employed. The employed value is used to correct a current value obtained through the first to fourth electrodes. The corrected current value is used to check the presence/absence of the target nucleic acid and to determine its amount. This applies to the straight flow channel regions from the second row.

With this arrangement, in the straight flow channel region of each row, the current value can be corrected to reflect the concentration distribution of the nucleic acid recognition bodies at each electrode position. When the value of the nucleic acid probe is compared with the corrected current value, highly accurate detection can be performed.

### Eighth Embodiment

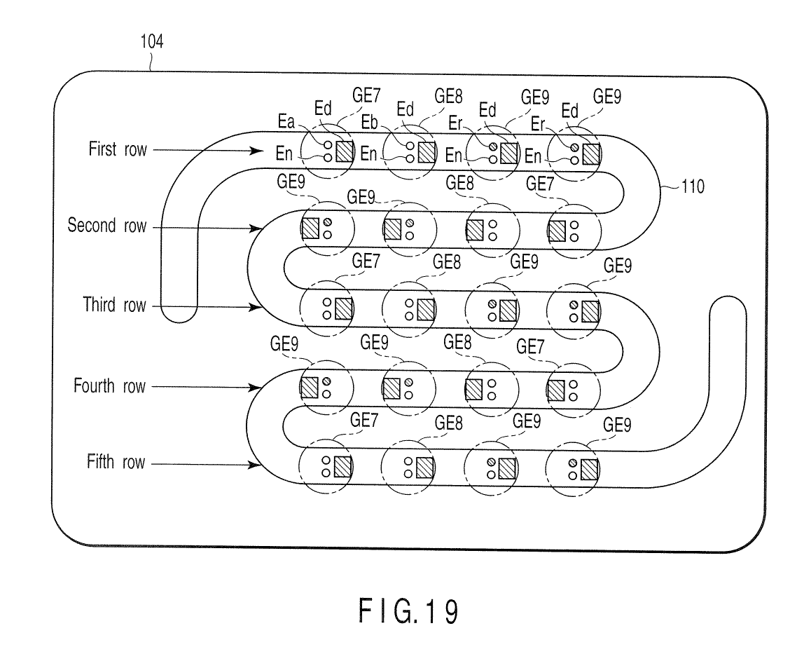

In a nucleic acid detection device according to the eighth embodiment, as shown in FIG. 19, in the first-row straight flow channel region, the first electrode is a probe electrode to which a nucleic acid probe having a sequence complimentary to a target nucleic acid is immobilized. The third electrode is a probe electrode to which a nucleic acid probe having a sequence uncomplimentary to the target nucleic acid is immobilized. The fifth and seventh electrodes are reference electrodes which are used for measuring a reference value. This applies to the straight flow channel regions from the second row. Concentration measurement electrodes Ed are arranged in the vicinities of the respective electrodes.

In other words, each straight flow channel region includes four electrode portions arranged with the same sequence pattern. In each straight flow channel region, an electrode portion GE7 which is located on the most upstream side has one probe electrode Ea to which nucleic acid probes (complimentary sequence) are immobilized and one electrode En which has no special function. An electrode portion GE8 which is located on the second upstream side has one probe electrode Eb to which nucleic acid probes (uncomplimentary sequence) are immobilized and one electrode En which has no special function. Each remaining electrode portion GE9 has one reference electrode Er and one electrode En which has no special function. Therefore, in an entire flow channel 110, the electrode portions are repeatedly arranged with the sequence pattern of GE7, GE8, GE9, and GE9 along the flow channel 110. The concentration measurement electrodes Ed are arranged near the electrodes in the respective electrode portions.

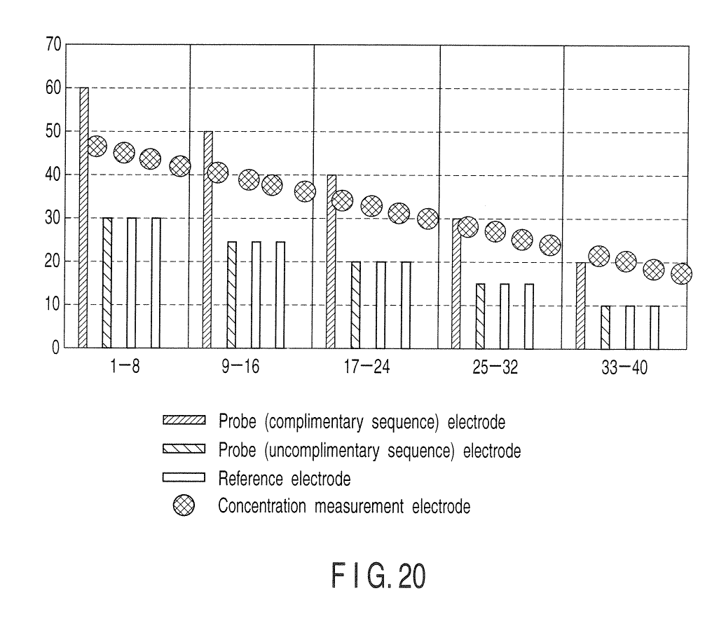

In the electrochemical response shown in FIG. 20, a value calculated on the basis of a value measured through a concentration measurement electrode arranged in the vicinity of each electrode is employed. The employed value is used to correct a current value obtained through each electrode. The corrected current value is used to check the presence/absence of the target nucleic acid and to determine its amount. This applies to the remaining rows.

With this arrangement, in each row, the current value can be corrected to reflect the concentration distribution of the nucleic acid recognition bodies at each electrode position. When the value of the nucleic acid probe is compared with the corrected current value, highly accurate detection can be performed.

### Example 1

An example that uses a full-automatic nucleic acid detection cassette according to the first embodiment will be described in detail.

1. Preparation of Nucleic Acid Detection Device

In the first-row straight flow channel region, nucleic acid probes (A) were immobilized to the first and second electrodes. Nucleic acid probes (B) were immobilized to the third and fourth electrodes. Reference value measurement nucleic acid probes (C) were immobilized to the seventh and eighth electrodes. The same process was performed for the straight flow channel regions from the second row. The probes were immobilized by dipping the surfaces of Au electrodes on a nucleic acid detection device in a solution of single stranded nucleic acid probe for 1 hr. The employed single stranded nucleic acid probes had the following sequences:

### A: gtttctgcac ccgga

### B: gtttctgcgc ccgga

### C: gacctgcttc tgact

2. Detection of Target Nucleic Acid

As the target nucleic acid, a PCR product was used. The sequence of the PCR product has a sequence complimentary to the single stranded nucleic acid (A). The sequence of the PCR product is as follows. Target Nucleic Acid: ggcctccgct ctcgcttcgc ctctttcacc ccgcgcccag ccccgccccg cgccgcgaag aaatgaaact cacagaccct gtgctgaggg cggctccggg cgcagaaacg aaacctagct

Samples each formed of a 2×SSC solution containing 1×10¹⁴ copy/ml of target nucleic acid as a final concentration were prepared. The nucleic acid probe immobilized Au electrodes fabricated in the preparation step were dipped in the respective solution samples to perform a hybridization reaction. The Au electrodes were then washed with a 0.2×SSC solution. After a solution containing 50 μM of Hoechst 33258 solution was flowed through the flow channel for 15 min, the oxidation current response of Hoechst 33258 molecule was measured. With the electrode to which the nucleic acid probe (A) was immobilized, a large signal increase was observed when compared to the reference electrode. With the electrode to which the nucleic acid probe (B) was immobilized, the signal increase was substantially the same level as that of the current value of the reference electrode. From this result, the target nucleic acid did not cause nonspecific adsorption to the nucleic acid probe (B) and had a sequence complimentary to the nucleic acid probe (A).

Additional advantages and modifications will readily occur to those skilled in the art. Therefore, the invention in its broader aspects is not limited to the specific details and representative embodiments shown and described herein. Accordingly, various modifications may be made without departing from the spirit and scope of the general inventive concept as defined by the appended claims and their equivalents.

What is claimed is:

1. A nucleic acid detection device to be used for electrochemical nucleic acid detection, comprising:
a flow channel which allows a solution containing a nucleic acid recognition body to flow therethrough;
at least two first electrode portions including a probe electrode to which at least one nucleic acid probe is immobilized; and
at least two second electrode portions including a reference electrode to be used for measuring a reference value, the first and second electrode portions being arranged in the flow channel to form a row along the flow channel, and one of the second electrode portions being adjacent to one of the first electrode portions.

2. A device according to claim 1, wherein at least one of the first electrode portions is located between the second electrode portions.

3. A device according to claim 1, wherein the first electrode portions are located on both sides of the second electrode portions.

4. A device according to claim 1, wherein at least one probe electrode of the first electrode portions includes a probe with a nucleic acid sequence which is different from that of a probe of at least another one of the probe electrodes of the first electrode portions.

5. A device according to claim 1, wherein the flow channel includes straight flow channel regions and at least one connection flow channel region which connects two adjacent straight flow channel regions, the at least two second electrode portions are respectively arranged in different straight flow channel regions, the at least two first electrode portions are respectively arranged in different straight flow channel regions, and at least two straight flow channel regions include at least one of the second electrode portions and at least one of the first electrode portions.

6. A device according to claim 1, wherein the first and second electrode portions are repeatedly arranged along the flow channel in accordance with a predetermined sequence pattern based on a difference in electrodes included in each of the first and second electrode portions.

7. A device according to claim 1, wherein the reference electrode comprises an electrode to which a nucleic acid probe for measuring the reference value is immobilized.

8. A nucleic acid detection device to be used for electrochemical nucleic acid detection, comprising:
a flow channel which allows a solution containing a nucleic acid recognition body to flow therethrough; and
electrode portions which are arranged in the flow channel to form a row along the flow channel, the electrode portions including a probe electrode to which at least one nucleic acid probe is immobilized and a reference electrode to be used for measuring a reference value.

9. A device according to claim 8, wherein at least one probe electrode of the electrode portions includes a probe with a nucleic acid sequence which is different from that of a probe of at least another one probe electrode of the electrode portions.

10. A device according to claim 8, wherein the electrode portions are repeatedly arranged along the flow channel in accordance with a predetermined sequence pattern based on a difference in electrodes included in each of the electrode portions.

11. A device according to claim 8, wherein the flow channel includes straight flow channel regions and at least one connection flow channel region which connects two adjacent straight flow channel regions, and some of the electrode portions are arranged in each of the straight flow channel regions.

12. A device according to claim 11, wherein one of the electrode portions is arranged near either one of an inlet and an outlet of the flow channel.

13. A device according to claim 8, wherein the reference electrode comprises an electrode to which a nucleic acid probe for measuring the reference value is immobilized.

14. A nucleic acid detection device to be used for electrochemical nucleic acid detection, comprising:
a flow channel which allows a solution containing a nucleic acid recognition body to flow therethrough;
at least two first electrode portions including a probe electrode to which at least one nucleic acid probe is immobilized;
at least one second electrode portion including a reference electrode to be used for measuring a reference value, the first and second electrode portions being arranged in the flow channel to form a row along the flow channel; and
at least two concentration measurement electrodes arranged in the flow channel to be used for measuring a relative value of a concentration distribution of the nucleic acid recognition body in the flow channel.

15. A device according to claim 14, wherein the first and second electrode portions are located between the concentration measurement electrodes.

16. A device according to claim 14, wherein the first and second electrode portions and the concentration measurement electrodes are repeatedly arranged along the flow channel in accordance with a predetermined sequence pattern.

17. A device according to claim 14, wherein the flow channel includes straight flow channel regions and at least one connection flow channel region which connects two adjacent straight flow channel regions, the first and second electrode portions are located in the straight flow channel regions, and the device includes at least one concentration measurement electrode for each of the straight flow channel regions.

18. A device according to claim 14, wherein the number of concentration measurement electrodes is equal to that of the first and second electrode portions, and the concentration measurement electrodes are located adjacent to the first and second electrode portions, respectively.

19. A device according to claim 14, wherein the concentration measurement electrodes comprise electrodes each having a layer of a blocking molecule comprising a normal chain organic molecule on a surface thereof.

20. A device according to claim 14, wherein the concentration measurement electrodes comprise electrodes each having a layer made of a nucleic acid molecule that interacts with the nucleic acid recognition body on a surface thereof.

21. A nucleic acid detection apparatus which performs electrochemical nucleic acid detection using a device according to claim 1, wherein nucleic acid detection is performed with reference to a numerical value which is calculated using a value measured through the reference electrode.

22. A nucleic acid detection apparatus which performs electrochemical nucleic acid detection using a device according to claim 5, wherein nucleic acid detection is performed with reference to a numerical value which is calculated using a value measured through the reference electrode.

23. A nucleic acid detection apparatus which performs electrochemical nucleic acid detection using a device according to claim 13, wherein nucleic acid detection is performed using a value which is obtained by correcting a value measured through either one of the probe electrode and the reference electrode by a value measured through either one of the concentration measurement electrodes.

24. A nucleic acid detection apparatus which performs electrochemical nucleic acid detection using a device according to claim 13, wherein nucleic acid detection is performed using a value which is obtained by correcting a value measured through the probe electrode and the reference electrode by a value measured through the concentration measurement electrodes.
